(19) United States
(12) Patent Application Publication
(10) Pub. No.: US 2021/0012886 A1
(43) Pub. Date: Jan. 14, 2021

(54) IMAGE PROCESSING APPARATUS, IMAGE PROCESSING METHOD, AND STORAGE MEDIUM

(71) Applicant: OLYMPUS CORPORATION, Tokyo (JP)

(72) Inventors: Akihiro KUBOTA, Tokyo (JP); Makoto KITAMURA, Tokyo (JP); Yamato KANDA, Tokyo (JP); Katsuyoshi TANIGUCHI, Tokyo (JP)

(73) Assignee: OLYMPUS CORPORATION, Tokyo (JP)

(21) Appl. No.: 16/936,503

(22) Filed: Jul. 23, 2020

Related U.S. Application Data
(63) Continuation of application No. PCT/JP2018/002490, filed on Jan. 26, 2018.

(51) Int. Cl. G16H 30/40; G06T 7/00

(52) U.S. Cl. CPC G16H 30/40; G06T 2207/30096; G06T 2207/10068; G06T 7/0012

(57) ABSTRACT

An image processing apparatus includes an input circuit to which an image acquired by medical equipment is sequentially inputted and a processor. The processor detects a lesion candidate in the image, discriminates the lesion candidate detected and outputs discrimination information, when two or more lesion candidates are detected in the image, selects a discrimination object lesion candidate which is a discrimination object based on information on distance between the medical equipment and the lesion candidates, and outputs position information and discrimination information of the discrimination object lesion candidate together with the image.

### CROSS REFERENCE TO RELATED APPLICATION

This application is a continuation application of PCT/JP2018/002490 filed on Jan. 26, 2018, the entire contents of which are incorporated herein by this reference

### BACKGROUND OF THE INVENTION

### 1. Field of the Invention

The present invention relates to an image processing apparatus, an image processing method and a storage medium.

### 2. Description of the Related Art

Conventionally, there has been proposed an image processing device which indicates a location of a lesion candidate included in a medical image.

Japanese Patent Application Laid-Open Publication No. 2004-135868 discloses an example of an abnormal shadow candidate detection processing system where an abnormal shadow candidate is detected from a medical image picked up by mammography or the like, malignancy confidence which indicates a degree of possibility that the abnormal shadow candidate is a malignant shadow is calculated and the malignancy confidence is displayed together with a marker which specifies a position, a shape and a size of the abnormal shadow candidate.

### SUMMARY OF THE INVENTION

According to one aspect of the present invention, an image processing apparatus includes: an input circuit to which an image acquired by medical equipment is sequentially inputted; and a processor, wherein the processor is configured to: detect a lesion candidate in the image; discriminate the lesion candidate detected and output discrimination information; when two or more lesion candidates are detected in the image, select a discrimination object lesion candidate which is a discrimination object based on information on distance between the medical equipment and the lesion candidates; and output position information and the discrimination information of the discrimination object lesion candidate together with the image.

According to another aspect of the present invention, an image processing method includes: sequentially inputting an image acquired by medical equipment; detecting a lesion candidate in the image; discriminating the lesion candidate detected and outputting discrimination information; calculating information on distance between the medical equipment and the lesion candidate; when two or more lesion candidates are detected in the image, selecting a discrimination object lesion candidate which is a discrimination object based on the information on distance; and outputting position information and the discrimination information of the discrimination object lesion candidate together with the image.

According to still another aspect of the present invention, a storage medium for storing an image processing program which is computer-readable and non-transitory, wherein the image processing program causes a computer to: sequentially input an image acquired by a medical equipment; detect a lesion candidate in the image; discriminate the lesion candidate detected and output discrimination information; acquire information on distance between the medical equipment and the lesion candidate; when two or more lesion candidates are detected in the image, select a discrimination object lesion candidate which is a discrimination object based on the information on distance; and output position information and the discrimination information of the discrimination object lesion candidate together with the image.

### BRIEF DESCRIPTION OF THE DRAWINGS

FIG. 1 is a block diagram showing one example of a configuration of an endoscope image processing apparatus according to a first embodiment of the present invention;

FIG. 2 is a block diagram showing one example of a configuration of an endoscope apparatus according to a second embodiment of the present invention;

FIG. 3 is a flowchart showing one example of a flow of an image processing of the endoscope apparatus according to the second embodiment of the present invention;

FIG. 4 is a view showing one example of a display image of the endoscope apparatus according to the second embodiment of the present invention;

FIG. 5 is a view for describing one example of parameters of lesion candidates detected by an endoscope apparatus according to a third embodiment of the present invention;

FIG. 6 is a block diagram showing one example of a configuration of an endoscope apparatus according to a fourth embodiment of the present invention;

FIG. 7 is a flowchart showing one example of a flow of an image processing of the endoscope apparatus according to the fourth embodiment of the present invention;

FIG. 8 is a view showing one example of a display image of the endoscope apparatus according to the fourth embodiment of the present invention;

FIG. 9 is a view showing one example of a display image of an endoscope apparatus according to a modification 1 of the fourth embodiment of the present invention;

FIG. 10 is a view showing one example of a display image of an endoscope apparatus according to a modification 2 of the fourth embodiment of the present invention;

FIG. 11 is a view showing one example of a display image of an endoscope apparatus according to a modification 3 of the fourth embodiment of the present invention;

FIG. 12 is a block diagram showing one example of a configuration of an endoscope apparatus according to a fifth embodiment of the present invention;

FIG. 13 is a flowchart showing one example of a flow of an image processing of the endoscope apparatus according to the fifth embodiment of the present invention;

FIG. 14 is a block diagram showing one example of a configuration of an endoscope apparatus according to a sixth embodiment of the present invention; and

FIG. 15 is a flowchart showing one example of a flow of an image processing of the endoscope apparatus according to the sixth embodiment of the present invention.

### DETAILED DESCRIPTION OF THE PREFERRED EMBODIMENTS

Hereinafter, embodiments of the present invention are described with reference to drawings.

### First Embodiment

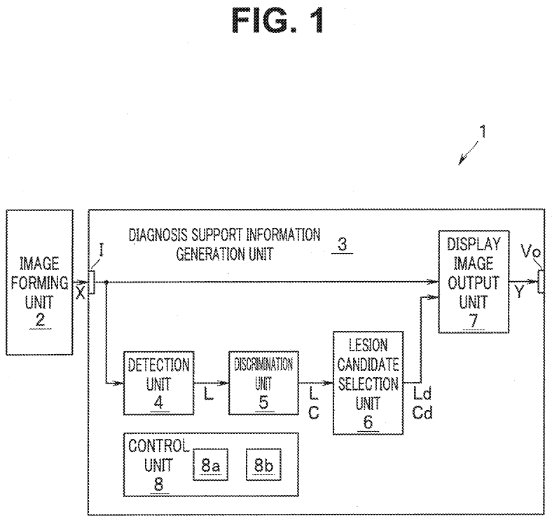

FIG. 1 is a block diagram showing one example of a configuration of an endoscope image processing apparatus 1 according to a first embodiment of the present invention.

The endoscope image processing apparatus 1 includes an image forming unit 2 and a diagnosis support information generation unit 3.

The image forming unit 2 is a device where an image pickup unit (not shown) disposed on a distal end of the image forming unit 2 is inserted into a subject, the subject is illuminated, and an endoscope image X (the endoscope image X being not limited to an endoscope image but any image acquired by medical equipment) is formed by picking up an image of the subject.

The diagnosis support information generation unit 3 is a device which forms a display image Y containing a discrimination object lesion candidate Ld and discrimination information Cd based on the endoscope image X, and outputs the display image Y. The diagnosis support information generation unit 3 includes an input unit I, a detection unit 4, a discrimination unit 5, a lesion candidate selection unit 6 and a display image output unit 7. The detection unit 4, the discrimination unit 5, the lesion candidate selection unit 6 and the display image output unit 7 are each formed of a circuit. However, each of such units may be formed of a processing unit, an operation of which is executed by a control unit 8.

The input unit I is a circuit which is connected between the image forming unit 2 and the detection unit 4. The endoscope image X is sequentially inputted from the image forming unit 2 to the input unit I, and the input unit I outputs the endoscope image X to the detection unit 4 and the display image output unit 7.

The detection unit 4 detects lesion candidates L in the endoscope image X.

The discrimination unit 5 discriminates the detected lesion candidates L and outputs discrimination information C.

When two or more lesion candidates L are detected in the endoscope image X, the lesion candidate selection unit 6 selects a discrimination object lesion candidate Ld which is a discrimination object from among the lesion candidates L, and outputs the discrimination object lesion candidate Ld and discrimination information Cd associated with the discrimination object lesion candidate Ld to the display image output unit 7.

The display image output unit 7 is a circuit which outputs a display image Y containing position information and the discrimination information Cd of the discrimination object lesion candidate Ld together with the endoscope image X.

The control unit 8 is a circuit which controls the respective units in the endoscope image processing apparatus 1. The control unit 8 includes a CPU 8a and a memory 8b. Functions of the control unit 8 are realized such that the CPU 8a reads programs of various processing units and various types of information stored in the memory 8b and executes the programs.

According to the embodiment, the endoscope image processing apparatus 1 can display the position information of the discrimination object lesion candidate Ld and the discrimination information Cd associated with the discrimination object lesion candidate Ld in a state where the observation of the endoscope image X does not become difficult.

### Second Embodiment

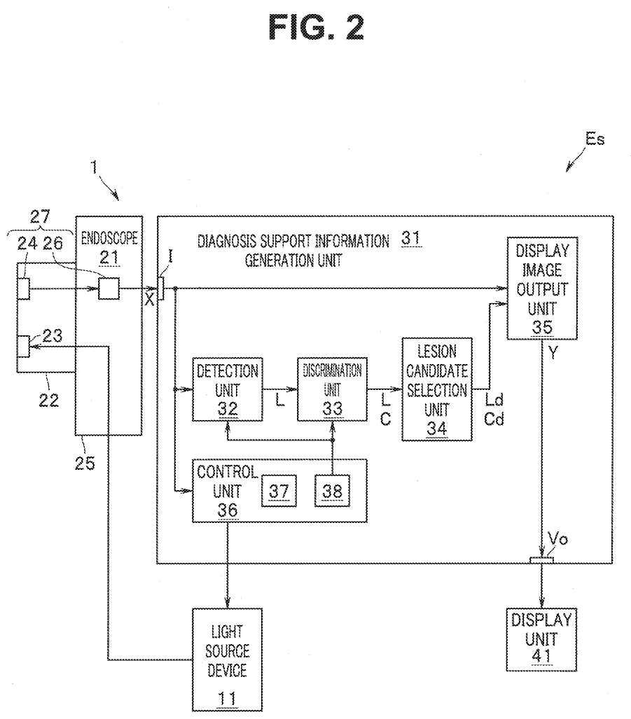

FIG. 2 is a block diagram showing one example of a configuration of an endoscope apparatus Es according to a second embodiment of the present invention. In the second embodiment, description of components equivalent to corresponding components in other embodiments and modifications is omitted.

The endoscope apparatus Es includes a light source device 11, an endoscope 21, a diagnosis support information generation unit 31 and a display unit 41. The light source device 11 is connected to each of the endoscope 21 and the diagnosis support information generation unit 31. The endoscope 21 is medical equipment and is connected to the diagnosis support information generation unit 31. The diagnosis support information generation unit 31 is connected to the display unit 41.

The endoscope 21 and the diagnosis support information generation unit 31 form an endoscope image processing apparatus 1.

The light source device 11 outputs illumination light to an illumination unit 23 mounted on a distal end portion of a scope 22 of the endoscope 21 under a control of the diagnosis support information generation unit 31. The light source device 11 can output a narrow-band light besides a white light in response to an instruction by a user through an operation unit not shown under a control of a control unit 36.

The endoscope 21 is configured to pick up an image of an inside of a subject. The endoscope 21 includes the scope 22, the illumination unit 23, an imager 24, a control unit 25 and an image processing unit 26.

The scope 22 is formed in an elongated shape so that the scope 22 can be inserted into the subject. Various tubes and signal lines not shown are inserted into the scope 22. The scope 22 is bendable in response to an inputting of an instruction by a user using the operation unit and a bending mechanism.

The illumination unit 23 is mounted on the distal end portion of the scope 22, and radiates illumination light inputted from the light source device 11 to the subject.

The imager 24 is mounted on the distal end portion of the scope 22. The imager 24 is formed of an image pickup device such as a CCD. The imager 24 picks up an image of the subject illuminated by the illumination unit 23 and outputs an image pickup signal to the control unit 25.

The control unit 25 is mounted on a proximal end portion of the scope 22, and is connected to the diagnosis support information generation unit 31. The control unit 25 includes the operation unit not shown, and can perform inputting of an instruction such as switching of illumination light radiated from the illumination unit 23 or bending of the scope 22.

The image processing unit 26 is provided to the control unit 25. The image processing unit 26 forms an endoscope image X by applying image processing such as gain adjustment, white balance adjustment, gamma correction, contour enhancement correction or zooming adjustment to an image pickup signal inputted from the imager 24, and outputs the endoscope image X to the diagnosis support information generation unit 31.

The imager 24 and the image processing unit 26 form an image forming unit 27. A part of image processing performed by the image forming unit 27 may be performed in the diagnosis support information generation unit 31.

The diagnosis support information generation unit 31 is a device which forms a display image Y based on the endoscope image X, and outputs the display image Y. The diagnosis support information generation unit 31 includes an input unit I, a detection unit 32, a discrimination unit 33, a lesion candidate selection unit 34, a display image output unit 35 and a control unit 36. The detection unit 32, the discrimination unit 33, the lesion candidate selection unit 34 and the display image output unit 35 are each formed of a circuit. However, each of such units may be formed of a processing unit, an operation of which is executed by the control unit 36 having a processor.

The input unit I is a circuit which is connected between the image forming unit 27 and the detection unit 32. The endoscope image X is sequentially inputted from the image forming unit 27 to the input unit I, and the input unit I outputs the endoscope image X to detection unit 32 and the display image output unit 35.

The detection unit 32 detects a plurality of lesion candidates L from the endoscope image X by performing predetermined detection processing. The detection unit 32 detects one or a plurality of lesion candidates L from the endoscope image X, and outputs the lesion candidates L to the discrimination unit 33.

In the predetermined detection processing, for example, predetermined contour extracting processing is performed based on a change amount between pixels arranged adjacently to each other. In the contour extracting processing, lesion candidates L are extracted by performing matching of extracted contours and model information read from a memory 38. The predetermined detection processing is not limited to such processing. For example, the predetermined detection processing may be performed based on calculation of colors or a spatial frequency of the endoscope image X. The predetermined detection processing may be processing where the lesion candidates L are detected by an arithmetic device which uses an artificial intelligence technique such as machine learning. The predetermined detection processing may also be performed by analyzing colors or spatial frequency by an arithmetic device which uses an artificial intelligence technique.

The lesion candidate L contains position information of the lesion candidate L. The position information is, for example, coordinates of a center position of the lesion candidate L in the endoscope image X.

The discrimination unit 33 discriminates the lesion candidates L by performing predetermined discrimination processing, and outputs discrimination information C which indicates seriousness of the lesion candidates L. The predetermined discrimination processing generates the discrimination information C based on classification information in accordance with NICE classification read from the memory 38, for example, for supporting a diagnosis of the lesion candidates L by a user. When the lesion candidates L are inputted from the detection unit 32, the discrimination unit 33 extracts images of the lesion candidates L, performs predetermined discrimination processing based on the extracted images, and outputs the lesion candidates L and the discrimination information C associated with the lesion candidates L to the lesion candidate selection unit 34. The predetermined discrimination processing is not limited to such processing, and may be performed by an arithmetic device which uses an artificial intelligence technique. Processing in the detection unit 32 and processing in the discrimination unit 33 may be collectively performed. When two or more lesion candidates L are contained in the endoscope image X, the lesion candidate selection unit 34 selects a discrimination object lesion candidate Ld which is a discrimination object. When the lesion candidates L and the discrimination information C associated with the lesion candidates L are inputted from the discrimination unit 33, the lesion candidate selection unit 34 selects the discrimination object lesion candidate Ld under a predetermined condition from among the lesion candidates L, and outputs the discrimination object lesion candidate Ld and the discrimination information Cd associated with the discrimination object lesion candidate Ld to the display image output unit 35.

As an example, the predetermined condition is set such that the lesion candidate L located at a position closest to a center position of the endoscope image X is selected as the discrimination object lesion candidate Ld.

In other words, the lesion candidate selection unit 34 selects the discrimination object lesion candidate Ld based on the endoscope image X. More specifically, the lesion candidate selection unit 34 selects the discrimination object lesion candidate Ld after the discrimination unit 33 outputs the discrimination information C. The lesion candidate selection unit 34 selects the discrimination object lesion candidate Ld based on the position information of the lesion candidates L.

The display image output unit 35 outputs a display image Y containing the position information and the discrimination information Cd of the discrimination object lesion candidate Ld together with the endoscope image X. More specifically, the display image output unit 35 outputs a display image Y where the position information of the discrimination object lesion candidate Ld is superposed on the endoscope image X, and the discrimination information Cd associated with the discrimination object lesion candidate Ld is superposed on a position different from the endoscope image X.

The control unit 36 is a circuit which controls respective units in the endoscope apparatus Es. The control unit 36 includes a CPU 37 which is a processor and the memory 38. Functions of the control unit 36 are realized by causing the CPU 37 to read programs and various types of information stored in the memory 38 and to execute the programs.

The control unit 36 detects a gain adjustment amount or brightness of the endoscope image X, and outputs a control signal so as to control a light emitting amount of the light source device 11.

The control unit 36 outputs a control signal, and controls a light source mode of the light source device 11.

The memory 38 is formed of, for example, a readable and writable storage element such as a flash ROM. In the memory 38, besides various types of data for controlling the endoscope apparatus Es or programs of various processing units, model information read by the detection unit 32, and classification information in accordance with NICE classification read by the discrimination unit 33 are also stored. The memory 38 may also be used as a working memory which temporarily preserves information on the endoscope image X and the like.

The display unit 41 is formed of a monitor which can display color images, for example. The display unit 41 is connected to an output terminal Vo by wire, and displays a display image Y inputted from the output terminal Vo. The display unit 41 may be connected to the diagnosis support information generation unit 31 by radio communication via a radio communication unit (not shown) of the diagnosis support information generation unit 31.

Next, a manner of operation of the endoscope apparatus Es according to the second embodiment is described.

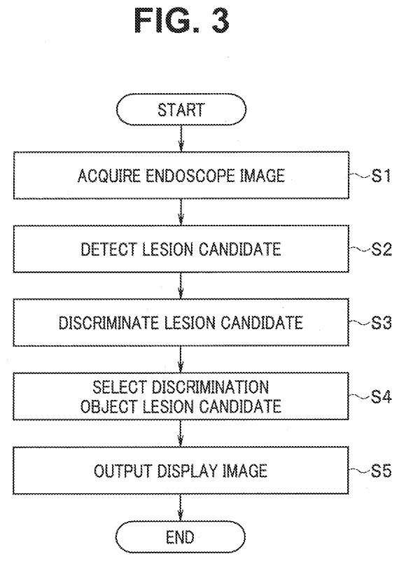

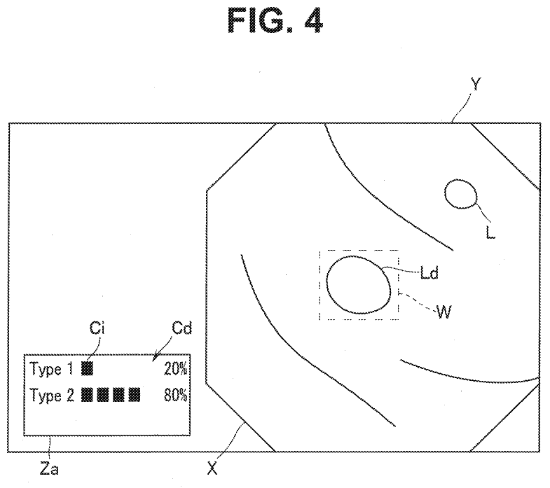

FIG. 3 is a flowchart showing one example of a flow of an image processing of the endoscope apparatus Es according to the second embodiment of the present invention. FIG. 4 is a view showing one example of the display image Y of the endoscope apparatus Es according to the second embodiment of the present invention.

An endoscope image X is acquired (S1). A user inserts the scope 22 into a subject, and radiates illumination light to the subject by driving the illumination unit 23. The image forming unit 27 picks up an image of the subject, acquires an endoscope image X by applying image processing to a picked up image, and outputs the endoscope image X to the diagnosis support information generation unit 31.

Lesion candidates L are detected (S2). The detection unit 32 detects the lesion candidates L from the endoscope image X by performing predetermined detection processing, and outputs the lesion candidates L to the discrimination unit 33.

The lesion candidates L are discriminated (S3). The discrimination unit 33 discriminates the lesion candidates L detected in S2 by performing predetermined discrimination processing. More specifically, the discrimination unit 33 reads classification information preliminarily stored in the memory 38. The classification information contains predetermined classification conditions and reference images in accordance with NICE classification. The discrimination unit 33 detects color based on a luminance value of an image, and detects blood vessels and a surface pattern by performing predetermined contour extracting processing. The discrimination unit 33 compares a detected result with predetermined classification conditions and reference images in accordance with NICE classification, calculates respective conformities of Type 1 to Type 3 which are classifications, and generates discrimination information C. The discrimination unit 33 outputs the lesion candidates L and discrimination information C associated with the lesion candidates L to the lesion candidate selection unit 34.

A discrimination object lesion candidate Ld is selected (S4). As shown in FIG. 4, the lesion candidate selection unit 34 selects the discrimination object lesion candidate Ld located close to a center position of the endoscope image X from among a plurality of lesion candidates L, and outputs the discrimination object lesion candidate Ld and discrimination information Cd to the display image output unit 35.

A display image Y is outputted (S5). The display image output unit 35 superposes position information of the discrimination object lesion candidate Ld on the display image Y. More specifically, the display image output unit 35 superposes a position display image W which indicates the position of the discrimination object lesion candidate Ld in the endoscope image X. In the example shown in FIG. 4, the position display image W is a quadrangular image which is superposed on the discrimination object lesion candidate Ld in a surrounding manner.

The display image output unit 35 superposes the discrimination information Cd on an information display region Za which differs from a region on which the endoscope image X is superposed. In the example shown in FIG. 4, the discrimination information Cd is indicated such that a conformity of Type 1 in accordance with NICE classification is 20% and conformity of Type 2 in accordance with NICE classification is 80%. The discrimination information Cd may contain an indicator Ci which performs an increase/decrease display corresponding to conformity.

In other words, in the endoscope image processing method according to the second embodiment, the endoscope image X is sequentially inputted to the input unit I. The detection unit 32 detects lesion candidates L in the endoscope image X. The discrimination unit 33 discriminates detected lesion candidates L and outputs discrimination information C. When two or more lesion candidates L are detected in the endoscope image X, the lesion candidate selection unit 34 selects a discrimination object lesion candidate Ld which is a discrimination object. The display image output unit 35 outputs position information and the discrimination information Cd of the discrimination object lesion candidate Ld together with the endoscope image X.

With such processing, the endoscope image processing apparatus 1 can select the discrimination object lesion candidate Ld located at the position closest to the center position of the endoscope image X from among the lesion candidates L. Further, the endoscope image processing apparatus 1 displays position information of the discrimination object lesion candidate Ld on the endoscope image X, and displays the discrimination information Cd at the position different from the endoscope image X in a state where the observation of the endoscope image X does not become difficult.

For example, when a user operates the endoscope 21 so as to display the target lesion candidate L at the center position, the endoscope image processing apparatus 1 displays position information of the discrimination object lesion candidate Ld located at the position closest to the center position of the endoscope image X from among the lesion candidates L, and displays the discrimination information Cd at the position different from the endoscope image X.

According to the second embodiment, the endoscope image processing apparatus 1 can display the position information and the discrimination information Cd of the discrimination object lesion candidate Ld in a state where the observation of the endoscope image X does not become difficult.

### Third Embodiment

In the first embodiment and the second embodiment, the discrimination object lesion candidate Ld is selected corresponding to position information of the lesion candidates L. However, the discrimination object lesion candidate Ld may be selected corresponding to information other than the position information.

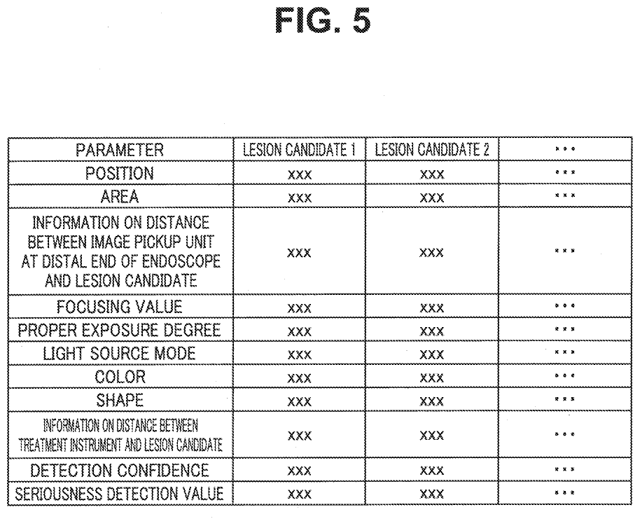

FIG. 5 is a view for describing one example of parameters of lesion candidates L detected by an endoscope apparatus Es according to a third embodiment of the present invention. In FIG. 5, “xxx” indicate parameter values. In FIG. 5, each of “lesion candidate 1” and “lesion candidate 2” indicates a plurality of detected lesion candidates L. In the third embodiment, description of components equivalent to corresponding components in other embodiments and modifications is omitted.

As shown in FIG. 5, as the respective parameter values, a detection unit 32 generates, besides respective position information on the lesion candidates L, area, information on distance between an image pickup unit at a distal end of an endoscope and the lesion candidate L, focusing value, proper exposure degree, light source mode, color, shape, information on distance between the lesion candidate L and a treatment instrument which medical equipment has, and detection confidence. Then, the detection unit 32 outputs the lesion candidates L containing the respective parameters to the discrimination unit 33.

The area is, for example, acquired by extracting a contour of the lesion candidate L by performing predetermined contour extracting processing and by calculating the area by performing a predetermined arithmetic operation based on the extracted contour.

Information on distance between the image pickup unit at the distal end of the endoscope and the lesion candidate L is, for example, calculated by performing a predetermined arithmetic operation based on a luminance value of pixels. The shorter the distance between the image pickup unit at the distal end of the endoscope and the lesion candidate L is, the larger the luminance value becomes. Although it is desirable that color of the pixels be red, the color of the pixels is not limited to red.

The information on distance between the image pickup unit at the distal end of the endoscope and the lesion candidate L may be calculated by performing a predetermined arithmetic operation based on a gain adjustment amount acquired by the control unit 36. The gain adjustment amount is an amount for correcting brightness of an endoscope image X. The shorter the distance between the image pickup unit at the distal end of the endoscope and the lesion candidate L is, the smaller the gain adjustment amount becomes.

The information on distance between the image pickup unit at the distal end of the endoscope and the lesion candidate L may be acquired by mounting a stereoscopic adopter on a distal end portion of a scope 22, by acquiring a stereoscopic image by the stereoscopic adopter, and by calculating information on distance by performing a predetermined arithmetic operation based on triangulation.

The information on distance between the image pickup unit at the distal end of the endoscope and the lesion candidate L may be acquired by extracting a contour of a blood vessel by performing predetermined contour extracting processing, by detecting a width of the blood vessel based on the extracted contour, and by calculating information on distance by performing a predetermined arithmetic operation based on the detected width of the blood vessel. The shorter the distance between the endoscope 21 and the lesion candidate L is, the larger the width of the blood vessel becomes.

The focusing value is, for example, calculated by performing a predetermined arithmetic operation based on spatial frequency of the endoscope image X. The higher the spatial frequency is, the larger the focusing value becomes.

The proper exposure degree is, for example, calculated by performing a predetermined arithmetic operation based on the endoscope image X and sensitivity of an image pickup device. The proper exposure degree may be acquired by detecting the exposure value of the lesion candidate L and by calculating the proper exposure degree based on divergence degree between the detected exposure value and the proper exposure value.

The light source mode is, for example, acquired by calculating an average luminance value of pixels of each color and by detecting the light source mode based on the average luminance value. For example, the light source mode is detected as a white light source mode when the average luminance value is a value within a first predetermined range, and is detected as a narrow-band light source mode when the average luminance value is a value within a second predetermined range. The detection unit 32 may not detect the light source mode based on an average luminance value, and may detect the light source mode by acquiring a parameter which indicates the light source mode from the control unit 36.

The color is, for example, detected by performing a predetermined arithmetic operation based on a luminance value of pixels of each color.

The shape is, for example, detected by performing a predetermined arithmetic operation based on predetermined contour extracting processing.

The information on distance between the lesion candidate L and the treatment instrument which medical equipment includes is, for example, acquired by extracting a treatment instrument image by performing a predetermined contour extracting processing, and by calculating the information on distance by performing a predetermined arithmetic operation based on the extracted treatment instrument image and the lesion candidate L.

The detection confidence is, for example, calculated by performing a predetermined arithmetic operation based on the lesion candidate L and model information.

The discrimination unit 33 calculates a seriousness calculation value by performing a predetermined arithmetic operation based on discrimination information C, and outputs the lesion candidate L containing the seriousness calculation value to the lesion candidate selection unit 34. The seriousness calculation value is calculated based on the discrimination information C.

The lesion candidate selection unit 34 selects a discrimination object lesion candidate Ld corresponding to respective values of position information, area, information on distance between an image pickup unit at a distal end of an endoscope and the lesion candidates L, focusing value, proper exposure degree, light source mode, color, shape, information on distance from the treatment instrument, detection confidence and seriousness calculation value which are detected or calculated by the detection unit 32 and the discrimination unit 33.

For example, the lesion candidate selection unit 34 may preferentially select the lesion candidate L having a large area as the discrimination object lesion candidate Ld. With such selection, when a user operates the endoscope 21 such that the target lesion candidate L is displayed in a large size, the lesion candidate selection unit 34 can select the discrimination object lesion candidate Ld having a large area from among the lesion candidates L.

The lesion candidate selection unit 34 may preferentially select the lesion candidate L located close to the image pickup unit at the distal end of the endoscope in distance as the discrimination object lesion candidate Ld based on the information on distance. With such selection, when a user brings the image pickup unit at the distal end of the endoscope close to the target lesion candidate L the lesion candidate selection unit 34 can select the discrimination object lesion candidate Ld having a short distance between the image pickup unit at the distal end of the endoscope and the lesion candidate L from among the lesion candidates L.

The lesion candidate selection unit 34 may preferentially select the lesion candidate L which falls on a focal point or matches an exposure as the discrimination object lesion candidate Ld. With such selection, when a user brings a focal point to the target lesion candidate L or matches an exposure at the target lesion candidate L, the lesion candidate selection unit 34 can select the discrimination object lesion candidate Ld which falls on a focal point or matches an exposure from among the lesion candidates L.

The lesion candidate selection unit 34 may exclude the lesion candidate L which has residue and halation based on the light source mode and the color. For example, in the light source mode of white light, the lesion candidate L which strongly exhibits white or yellow is excluded from the discrimination object lesion candidate Ld. The lesion candidate selection unit 34 may exclude the lesion candidate L which has a halation from the discrimination object lesion candidate Ld based on the detected shape.

The lesion candidate selection unit 34 may preferentially select the lesion candidate L having a shape close to a circle as the discrimination object lesion candidate Ld based on the detected shape. With such selection, when a user operates the endoscope 21 so as to pick up an image of a front side of the target lesion candidate L, the lesion candidate selection unit 34 can select the discrimination object lesion candidate Ld, the image of the front side of which is picked up, from among the lesion candidates L.

The lesion candidate selection unit 34 may preferentially select the lesion candidate L located close to the treatment instrument as the discrimination object lesion candidate Ld. With such selection, when a user brings the treatment instrument close to the target lesion candidate L, the lesion candidate selection unit 34 can select the discrimination object lesion candidate Ld having a short distance between the treatment instrument and the lesion candidate L from among the lesion candidates L.

The lesion candidate selection unit 34 may preferentially select the lesion candidate L having high detection confidence as the discrimination object lesion candidate Ld.

The lesion candidate selection unit 34 may preferentially select the lesion candidate L having a high seriousness calculation value as the discrimination object lesion candidate Ld.

For example, the lesion candidate selection unit 34 may select the discrimination object lesion candidate Ld based on an evaluation value by calculating the evaluation value by performing weighting with respect to respective values of position information, area, information on distance between the image pickup unit at the distal end of the endoscope and the lesion candidate L focusing value, proper exposure degree, light source mode, color, shape, information on distance between the treatment instrument and the lesion candidate L detection confidence and seriousness calculation value.

In other words, the lesion candidate selection unit 34 selects the discrimination object lesion candidate Ld based on the size of the lesion candidate L. The lesion candidate selection unit 34 selects the discrimination object lesion candidate Ld based on the information on distance between the image pickup unit at the distal end of the endoscope and the lesion candidates L. The lesion candidate selection unit 34 selects the discrimination object lesion candidate Ld based on the color of the lesion candidate L. The lesion candidate selection unit 34 selects the discrimination object lesion candidate Ld based on the shape of the lesion candidate L. The lesion candidate selection unit 34 selects the discrimination object lesion candidate Ld based on the information on distance between the treatment instrument and the lesion candidate L which appear on the endoscope image X. The lesion candidate selection unit 34 selects the discrimination object lesion candidate Ld based on the detection confidence of the lesion candidate L. The lesion candidate selection unit 34 selects the discrimination object lesion candidate Ld based on the seriousness calculation value which is calculated based on the discrimination information C. The lesion candidate selection unit 34 calculates the evaluation value by performing weighting with respect to a plurality of parameters which are acquired from the lesion candidates L and the discrimination information C, and selects the discrimination object lesion candidate Ld based on the evaluation value.

According to the third embodiment, the endoscope image processing apparatus 1 can select the discrimination object lesion candidate Ld which attracts attention of a user at a higher level, and can display the position information and the discrimination information Cd of the discrimination object lesion candidate Ld in a state where the observation of the endoscope image X does not become difficult.

### Fourth Embodiment

In the first embodiment to the third embodiment, the endoscope image processing apparatus 1 displays the discrimination object lesion candidate Ld from among the lesion candidates L. However, an endoscope image processing apparatus 1 may notify that a plurality of lesion candidates L are detected.

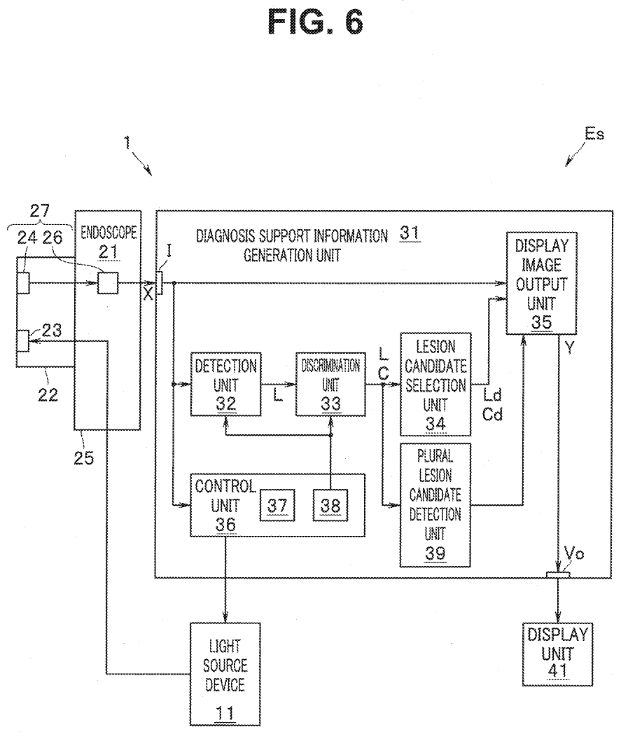

FIG. 6 is a block diagram showing one example of a configuration of an endoscope apparatus Es according to a fourth embodiment of the present invention. In the fourth embodiment, description of components equivalent to corresponding components in other embodiments and modifications is omitted.

The endoscope image processing apparatus 1 includes a plural lesion candidate detection unit 39.

The plural lesion candidate detection unit 39 detects that lesion candidates L exist in plural. The plural lesion candidate detection unit 39, when the lesion candidates L inputted from a discrimination unit 33 exist in plural, outputs flag information indicating that the lesion candidates L exist in plural to a lesion candidate selection unit 34. The plural lesion candidate detection unit 39 is formed of a circuit, but may be formed of a processing unit, an operation of which is executed by the control unit 36.

When the lesion candidates L exist in plural, the lesion candidates L other than the discrimination object lesion candidate Ld are not displayed. When the flag information indicating that the lesion candidates L exist in plural is inputted from the plural lesion candidate detection unit 39, the display image output unit 35 outputs a notification image F1 indicating that non-displayed lesion candidates L exist in such a manner that the notification image F1 is superposed on a display image Y.

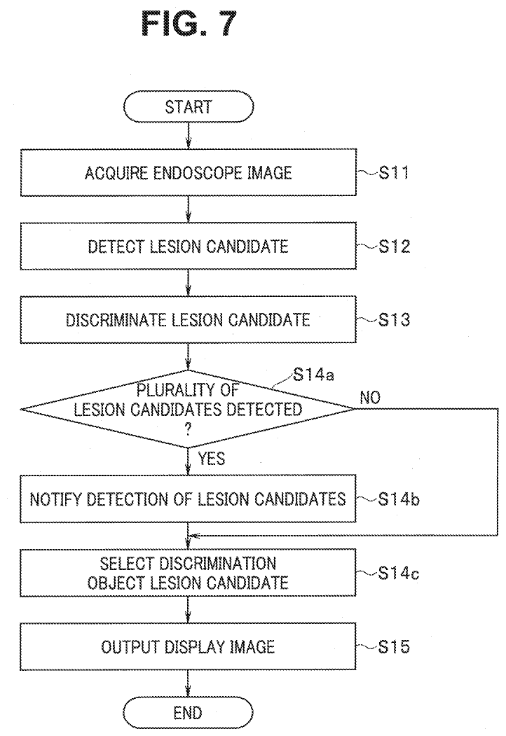

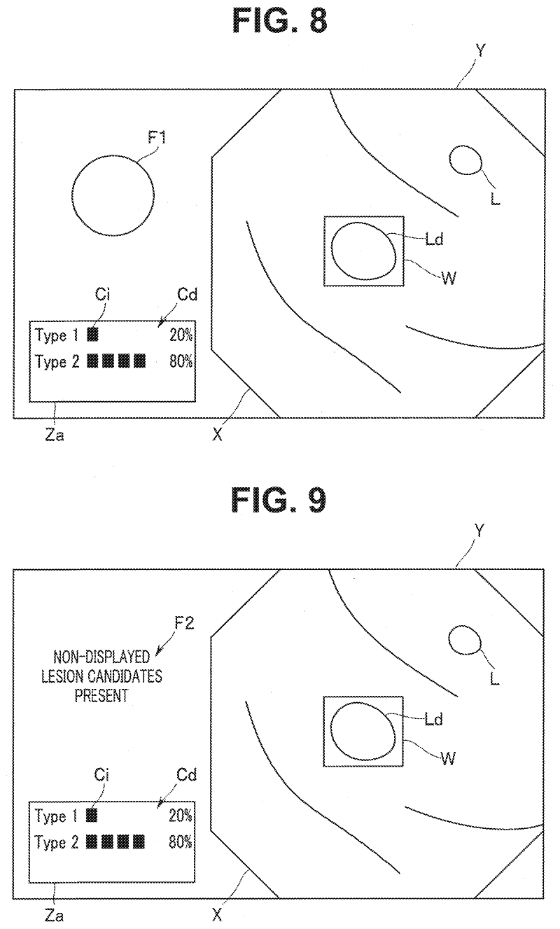

FIG. 7 is a flowchart showing one example of a flow of an image processing of the endoscope apparatus Es according to the fourth embodiment of the present invention. FIG. 8 is a view showing one example of a display image Y of the endoscope apparatus Es according to the fourth embodiment of the present invention.

S11 to S13 are equivalent to S1 to S3 and hence, the description of S11 to S13 is omitted.

Whether or not a plurality of lesion candidates L are detected is determined (S14a). The plural lesion candidate detection unit 39 determines whether or not the plurality of lesion candidates L are inputted from the discrimination unit 33. When the plurality of lesion candidates L are inputted (S14a: YES), processing advances to S14b. On the other hand, the plurality of lesion candidates L are not inputted but one lesion candidate L is inputted (S14a: NO), the processing advances to S14c.

In S14b, the notification is transmitted to the display image Y. The plural lesion candidate detection unit 39 instructs the display image output unit 35 to perform notification of plural detection. As shown in FIG. 8, the display image output unit 35 superposes the notification image F1 formed of a circular icon on the display image Y.

S14c and S15 are equivalent to S4 and S5 and hence, the description of S14c and S15 is omitted.

According to the fourth embodiment, the endoscope image processing apparatus 1 can notify that the non-displayed lesion candidate L exists, and can display position information and the discrimination information Cd of the discrimination object lesion candidate Ld in a state where the observation of the endoscope image X does not become difficult.

### (Modification 1 of Fourth Embodiment)

FIG. 9 is a view showing one example of a display image Y of an endoscope apparatus Es according to a modification 1 of the fourth embodiment of the present invention. In the modification 1 of the fourth embodiment, description of components equivalent to corresponding components in other embodiments and modifications is omitted.

In the fourth embodiment, the notification image F1 is formed of a circular icon. However, as shown in FIG. 9, a notification image F2 may be formed of a message “non-displayed lesion candidates L exist” which notifies that the non-displayed lesion candidates L exist.

### (Modification 2 of Fourth Embodiment)

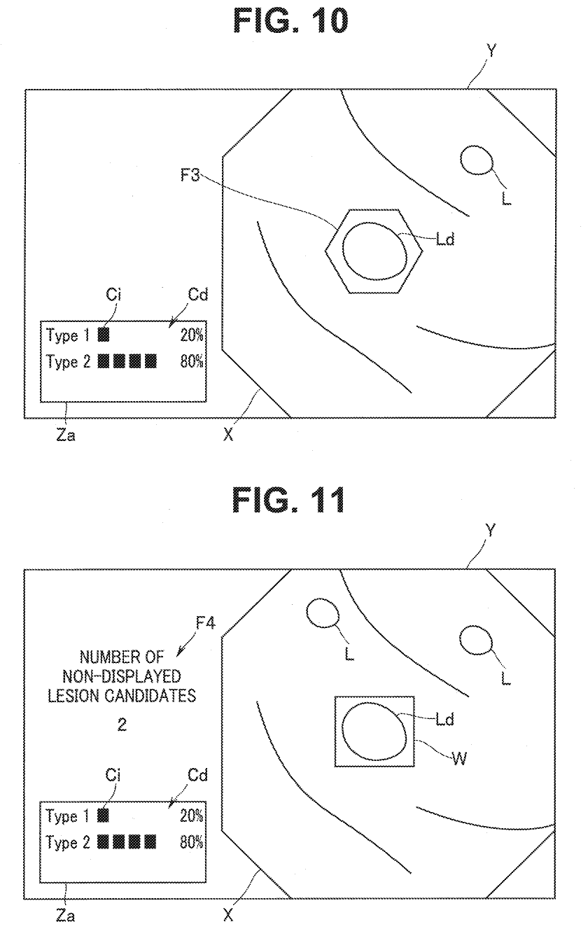

FIG. 10 is a view showing one example of a display image Y of an endoscope apparatus Es according to a modification 2 of the fourth embodiment of the present invention. In the modification 2 of the fourth embodiment, description of components equivalent to corresponding components in other embodiments and modifications is omitted.

In the fourth embodiment and the modification 1 of the fourth embodiment, the notification image F1 is formed of a circular icon, and the notification image F2 is formed of a message. However, a notification image F3 may be formed of an image which is superposed on the discrimination object lesion candidate Ld in a surrounding manner.

When the non-displayed lesion candidate L does not exist, a position display image W having a quadrangular pattern is superposed on the discrimination object lesion candidate Ld in a surrounding manner. As shown in FIG. 10, when the non-displayed lesion candidate L exists, the position display image W is not displayed but the notification image F3 having a hexagonal pattern which differs from the position display image W is superposed on the discrimination object lesion candidate Ld in a surrounding manner. The notification image F3 may differ from the position display image W in color.

### (Modification 3 of Fourth Embodiment)

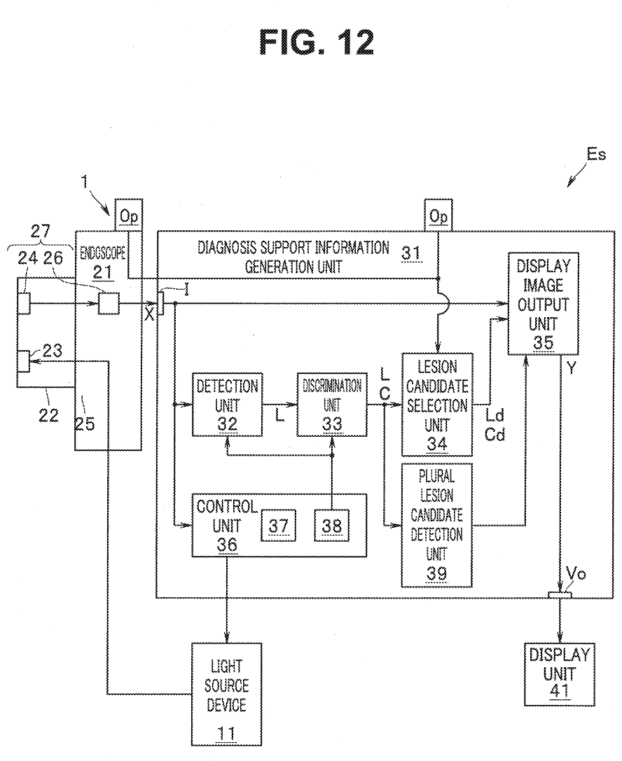

FIG. 11 is a view showing one example of a display image Y of an endoscope apparatus Es according to a modification 3 of the fourth embodiment of the present invention. In the modification 3 of the fourth embodiment, description of components equivalent to corresponding components in other embodiments and modifications is omitted.

In the fourth embodiment and the modifications 1 and 2 of the fourth embodiment, the notification image F1 is formed of a circular icon, the notification image F2 is formed of a message, and the notification image F3 is formed of an image which is superposed on the discrimination object lesion candidate Ld in a surrounding manner. However, a notification image F4 may be outputted by superposing the number of non-displayed lesion candidates L.

In other words, a display image output unit 35, when non-displayed lesion candidates L other than the discrimination object lesion candidate Ld exist in an endoscope image X, superposes the number of non-displayed lesion candidates L on the display image Y.

FIG. 11 is an example of the notification image F4 indicating that two non-displayed lesion candidates L exist.

According to the fourth embodiment and the modifications 1 to 3 of the fourth embodiment, the display image output unit 35, when the non-displayed lesion candidates L other than the discrimination object lesion candidate Ld exist in the endoscope image X, superposes the notification images F1, F2, F3 and F4 on the display image Y.

### Fifth Embodiment

In the first to fourth embodiments and the modifications of the fourth embodiment, the lesion candidate selection unit 34 may select the discrimination object lesion candidate Ld from among the lesion candidates L based on the endoscope image X. However, the discrimination object lesion candidate Ld may be selected based on inputting of an instruction by a user.

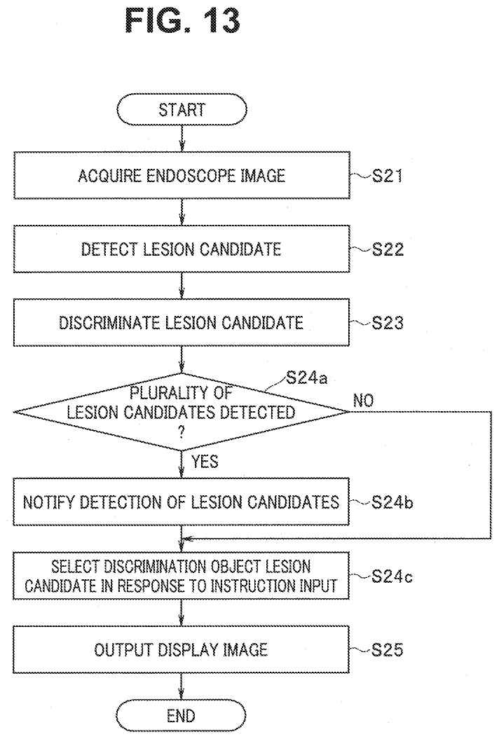

FIG. 12 is a block diagram showing one example of a configuration of an endoscope apparatus Es according to a fifth embodiment of the present invention. FIG. 13 is a flowchart showing one example of a flow of an image processing of the endoscope apparatus Es according to the fifth embodiment of the present invention. In the fifth embodiment, description of components equivalent to corresponding components in other embodiments and modifications is omitted.

An endoscope 21 and a diagnosis support information generation unit 31 each include an instruction input unit Op. The instruction input unit Op includes, for example, an operation switch.

S21 to S24b and S25 are equivalent to S1 to S14b and S15 and hence, the description of S21 to S24b and S25 is omitted.

A lesion candidate selection unit 34 selects the discrimination object lesion candidate Ld corresponding to inputting of an instruction using the instruction input unit Op (S24c).

According to the fifth embodiment, an endoscope image processing apparatus 1 can select the discrimination object lesion candidate Ld in response to inputting of an instruction by a user, and can display position information and the discrimination information Cd of the discrimination object lesion candidate Ld in a state where the observation of the endoscope image X does not become difficult.

### Sixth Embodiment

In the first to fourth embodiments and the modifications of the fourth embodiment, processing advances in order of the detection unit 32, the discrimination unit 33 and the lesion candidate selection unit 34. However, the processing may advance in order of a detection unit 32, a lesion candidate selection unit 34a and a discrimination unit 33a.

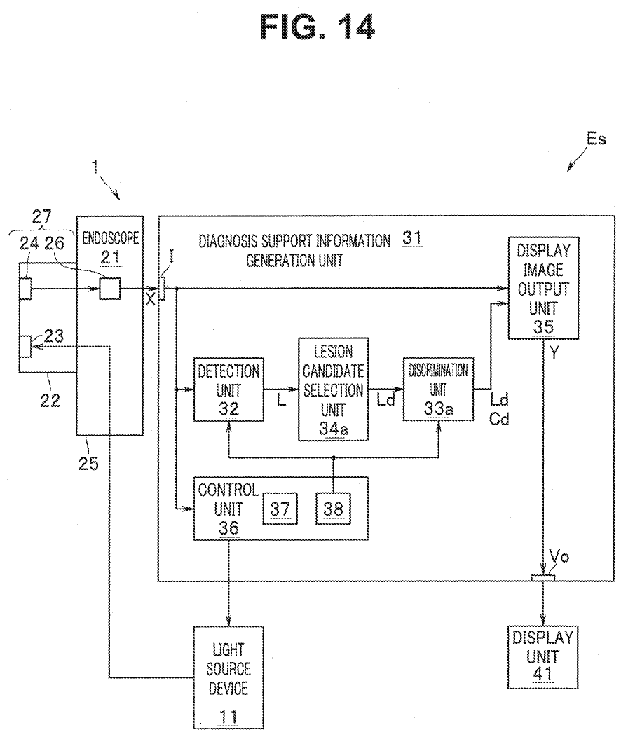

FIG. 14 is a block diagram showing one example of a configuration of an endoscope apparatus according to a sixth embodiment of the present invention. In the sixth embodiment, description of components equivalent to corresponding components in other embodiments and modifications is omitted.

As shown in FIG. 14, the detection unit 32 performs predetermined detection processing, and outputs lesion candidates L to the lesion candidate selection unit 34a. The lesion candidate selection unit 34a selects a discrimination object lesion candidate Ld from among the lesion candidates L, and outputs the discrimination object lesion candidate Ld to the discrimination unit 33a. The discrimination unit 33a generates discrimination information Cd indicating seriousness of the discrimination object lesion candidate Ld by performing predetermined discrimination processing based on the discrimination object lesion candidate Ld, and outputs the discrimination object lesion candidate Ld and the discrimination information Cd to a display image output unit 35.

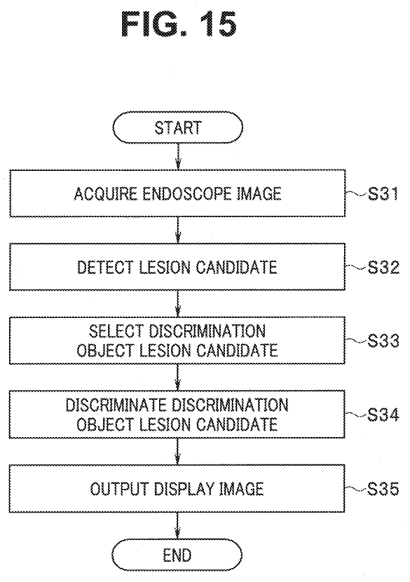

FIG. 15 is a flowchart showing one example of a flow of an image processing of the endoscope apparatus Es according to the sixth embodiment of the present invention.

S31 and S32 are equivalent to S1 and S2 and hence, the description of S31 and S32 is omitted.

The discrimination object lesion candidate Ld is selected (S33). The lesion candidate selection unit 34a selects the discrimination object lesion candidate Ld, and outputs the discrimination object lesion candidate Ld to the discrimination unit 33a.

The discrimination object lesion candidate Ld is discriminated (S34). The discrimination unit 33a discriminates the discrimination object lesion candidate Ld, and outputs the discrimination object lesion candidate Ld and the discrimination information Cd to the display image output unit 35.

S35 is equivalent to S5 and hence, the description of S35 is omitted.

In other words, after the discrimination object lesion candidate Ld is selected by the lesion candidate selection unit 34a, the discrimination unit 33a outputs the discrimination information Cd of the discrimination object lesion candidate Ld by performing predetermined discrimination processing.

According to the embodiment, the endoscope image processing apparatus 1 can reduce an arithmetic operation amount by selecting the discrimination object lesion candidate Ld before predetermined discrimination processing is performed by the discrimination unit 33a, and can display position information and the discrimination information Cd of the discrimination object lesion candidate Ld in a state where the observation of the endoscope image X does not become difficult.

In the embodiments and the modifications, the image forming unit 2, 27 and the diagnosis support information generation unit 3, 31 are formed as separate bodies. However, the image forming unit 2, 27 and the diagnosis support information generation unit 3, 31 may be formed as an integral body.

In the embodiments and the modifications, as one example, the description has been made with respect to an example where the discrimination unit 33, 33a outputs discrimination information C, Cd in accordance with NICE classification. However, the present invention is not limited to such an example. For example, the discrimination unit 33, 33a may output discrimination information C in accordance with medical classification such as Hiroshima classification or JNET classification.

In the specification, each “unit” does not always correspond to specific hardware or software routine on a one-to-one basis. Accordingly, in the specification, the embodiments have been described by estimating imaginary circuit blocks (units) having the respective functions of the embodiments. Further, all or some of the respective “units” in the embodiments may be realized by a software. Further, respective steps in each process of the embodiments may be performed in such a manner that the order of performing the steps is changed, a plurality of steps are performed simultaneously, or the order of steps is changed each time the image processing apparatus is operated so long as such modifications conform with the characteristic features of the embodiments.

For example, functions of the endoscope image processing apparatus 1 may be realized by causing the CPU 8a, 37 to execute a code stored in the memory 8b, 38.

In other words, the endoscope image processing apparatus 1 includes an endoscope image processing program for causing a computer to execute: a code for detecting the lesion candidates L in the endoscope image X; a code for discriminating the detected lesion candidates L and outputting discrimination information C; when two or more lesion candidates L are detected in the endoscope image X, a code for selecting a discrimination object lesion candidate Ld which is a discrimination object; and a code for outputting position information and the discrimination information Cd of the discrimination object lesion candidate Ld together with the endoscope image X. As described previously, provided that an image is an image acquired by medical equipment, the image is not limited to an endoscope image. Accordingly, a program which the endoscope image processing apparatus 1 includes is not limited to the endoscope image processing program, and it is sufficient that the program is an image processing program for processing an image acquired by medical equipment.

The present invention is not limited to the above-mentioned embodiments, and various changes, modifications and the like are conceivable without departing from the gist of the present invention.

What is claimed is:

1. An image processing apparatus comprising:
an input circuit to which an image acquired by medical equipment is sequentially inputted; and
a processor, wherein
the processor is configured to:
detect a lesion candidate in the image;
discriminate the lesion candidate detected and output discrimination information;
when two or more lesion candidates are detected in the image, select a discrimination object lesion candidate which is a discrimination object based on information on distance between the medical equipment and the lesion candidates; and
output position information and the discrimination information of the discrimination object lesion candidate with the image.

2. The image processing apparatus according to claim 1, wherein
the medical equipment is an endoscope, and
the processor is configured to select the discrimination object lesion candidate based on information on distance between the endoscope and the lesion candidates.

3. The image processing apparatus according to claim 1, wherein
the medical equipment includes a treatment instrument, and
the processor is configured to:
detect information on distance between the treatment instrument and the lesion candidates based on the treatment instrument and the lesion candidates in the image; and
select the discrimination object lesion candidate based on the information on distance.

4. An image processing method comprising:
sequentially inputting an image acquired by medical equipment;
detecting a lesion candidate in the image;
discriminating the lesion candidate detected and outputting discrimination information;
calculating information on distance between the medical equipment and the lesion candidate;
when two or more lesion candidates are detected in the image, selecting a discrimination object lesion candidate which is a discrimination object based on the information on distance; and
outputting position information and the discrimination information of the discrimination object lesion candidate with the image.

5. The image processing method according to claim 4, wherein in a case where the medical equipment is an endoscope,
information on distance between the endoscope and the lesion candidates is acquired, and
the discrimination object lesion candidate is selected based on the information on distance.

6. The image processing method according to claim 4, wherein in a case where the medical equipment includes a treatment instrument,
information on distance between the treatment instrument and the lesion candidates is calculated based on the treatment instrument and the lesion candidates in the image, and
the discrimination object lesion candidate is selected based on the information on distance.

7. A storage medium for storing an image processing program which is computer-readable and non-transitory, wherein
the image processing program causes a computer to:
sequentially input an image acquired by a medical equipment;
detect a lesion candidate in the image;
discriminate the lesion candidate detected and output discrimination information;
acquire information on distance between the medical equipment and the lesion candidate;
when two or more lesion candidates are detected in the image, select a discrimination object lesion candidate which is a discrimination object based on the information on distance; and
output position information and the discrimination information of the discrimination object lesion candidate with the image.

8. The storage medium according to claim 7, wherein in a case where the medical equipment is an endoscope,
the image processing program causes the computer to:
calculate information on distance between the endoscope and the lesion candidate; and
select the discrimination object lesion candidate based on the information on distance.

9. The storage medium according to claim 7, wherein in a case where the medical equipment includes a treatment instrument,
the image processing program causes the computer to:
calculate information on distance between the treatment instrument and the lesion candidate based on the treatment instrument and the lesion candidates in the image; and
select the discrimination object lesion candidate based on the information on distance.
